(19) United States
(12) Patent Application Publication
(10) Pub. No.: US 2021/0265047 A1
(43) Pub. Date: Aug. 26, 2021

(54) AUTOMATIC USER PROVISIONING, SUCH AS THE AUTOMATIC CREATION OF PROVIDER PROFILES IN AN ELECTRONIC MEDICAL RECORD SYSTEM

(71) Applicant: Providence St. Joseph Health, Seattle, WA (US)

(72) Inventors: Datla Anjaneya Raju, Portland, OR (US); Jan Rapp, Tigard, OR (US); Jose C. Galindo, Pasco, WA (US); Paul A. Cooke, Sammamish, WA (US)

(21) Appl. No.: 16/795,707

(22) Filed: Feb. 20, 2020

(51) Int. Cl. G16H 40/20; G16H 10/60; G06F 9/54

(52) U.S. Cl. CPC G16H 40/20; G06F 9/54; G16H 10/60

(57) ABSTRACT

A facility provisions a worker in a software system. The facility retrieves from an identity management system information about the worker. The facility determines, based upon the information retrieved from the identity management system, that a user profile should be created for the worker in a software system. The facility further determines, based upon the retrieved information, a set of user profile fields that should be populated for the worker in the software system. The facility causes a user profile to be created for the worker in the software system. For each of the determined set of user profile fields, the facility maps between the user profile field and a field among information maintained for the worker by the identity management system, and causes the determined field of the created user profile to be populated with the mapped field among information maintained for the worker by the identity management system.

### BACKGROUND

In a workplace, such as an office or a hospital, it is common for one or more software packages or online services to be provided by the employer to facilitate the work done by workers.

In many cases, these software packages or online services maintain per-user accounts or profiles. For example, an email client maintains an account for each email user that stores email messages received and sent by the user, address book information for the user, and credentials needed by the user to use the email client and access his or her account.

As another example, a particular employer may use payroll software to manage payroll payments to workers. The payroll software has a profile for each user containing information such as name, social security number, salary level, number of income tax exemptions claimed, and a history of payments made and taxes withheld.

As a third example, a hospital or group of hospitals may use an electronic medical record or electronic health record system (“EMR”) in its operations. The EMR maintains a provider profile for the medical providers (e.g., doctors, nurses, nursing assistants, physical and occupational therapists, and radiographers) who are employed by or otherwise operate in the hospital(s). Information stored in such a provider profile can include demographic, licensure, scheduling, billing, ordering, admitting, and attending information about each provider.

When a new worker is hired, it is typical for information technology and/or human resources workers to manually create accounts and profiles for the new worker for any software package or online service that's needed to facilitate the worker's work and effectively manage the worker. This process is sometimes called “provisioning.”

### BRIEF DESCRIPTION OF THE DRAWINGS

FIG. 1 is a block diagram showing some of the components typically incorporated in at least some of the computer systems and other devices on which the facility operates.

FIG. 2 is a data flow diagram showing a process performed by the facility in some embodiments in order to create provider profiles.

FIGS. 3A-B is a flow diagram showing a process performed by the facility in some embodiments to create a provider profile.

FIG. 4 is a table diagram showing sample contents of a provider profile creation business logic table used by the facility in some embodiments.

FIG. 5 is a table diagram showing sample contents of a field mapping table used by the facility in some embodiments to map between fields of the IMS and the EMRS.

### DETAILED DESCRIPTION

The inventors have identified disadvantages with conventional approaches to performing worker provisioning. In particular, establishing worker profiles and accounts manually, such as by typing or copying-and-pasting information about a new worker into a new profile or new account form displayed by the software package or online service is error-prone, and is often experienced as unrewarding work. Further, manual provisioning can take an unreasonably long time—such as weeks—during which a new worker must find a way to do his or her job without access to resources generally deemed to be important tools. This is particularly true in workplaces where: the flow of new workers is significant; the number of profiles and accounts that must be created for each worker is large; and/or too few people are assigned to create profiles and accounts, or the people assigned to create profiles and accounts are assigned additional responsibilities that compete with this task.

In the case of hospitals and other healthcare organizations, the inventors have discerned that having a new provider for whom an EMR provider profile has not been created interferes with the provider being scheduled to serve patients; the healthcare organization billing for the provider's services; the provider supervising others; etc. Thus, despite having hired, begun paying, and otherwise initiated a new provider, the healthcare organization is largely deprived of their assistance, which may affect the quality and volume of care provided to patients overall by the healthcare organization, provider cost-efficiency, etc.

In response to the inventors' recognition of these disadvantages, they have conceived and reduced to practice a software and/or hardware facility for automatically provisioning new providers in an EMR or other system (“the facility”). For example, in some embodiments, the facility automatically adds Schedulable Epic Resources (“SERs”) to the EPIC EMR system from Epic Systems Corporation. In various embodiments, the facility provides similar functionality for a variety of other EMR systems, including, for example, CERNER ELECTRONIC HEALTH RECORD from Cerner Corporation, ECLINICALWORKS EMR from eClinicalWorks, OPENEMR from OpenEMR Foundation, Inc., and PRACTICE FUSION EHR from Practice Fusion, Inc.

In some embodiments, the facility accesses information about every worker stored in a worker identity management system, such as THE SAILPOINT IDENTITYIQ system from Sailpoint Technologies Holdings, Inc. (Identity management systems are sometimes referred to herein as “IM systems,” “IMSs,” or “IMs.”) In various embodiments, the facility uses a variety of other IM systems, including, for example, MICROSOFT AZURE ACTIVE DIRECTORY from Microsoft Corporation, ORACLE IDENTITY CLOUD MANAGEMENT from Oracle Corporation, WORKFORCE IDENTITY from Okta, Inc., and INTELLIGENT IDENTITY PLATFORM from Ping Identity Holding Corporation. In various embodiments, populating the IMS is already a business process of the organization employing the workers and/or is performed automatically or semi-automatically; some or all of the information stored in the IMS that is used by the facility is used in the IMS for other purposes; etc.

In particular, for each worker, the facility uses business rules to determine from information about the worker's role stored in the IM system whether a provider profile should be created for the worker in the EMR system. In cases where the facility determines that the a provider profile should be created for the worker in the EMR system, the facility uses business rules to identify, again based on information about the worker's role, which fields of the provider profile should be populated for the worker. The facility then uses mappings from IMS fields to the identified EMRS fields to generate field/value pairs for the identified EMRS fields for the worker from information stored about the worker in the IMS, and calls the EMRS to create a new provider profile having the generated field/value pairs. The EMRS returns a provider profile identifier for the created provider profile. The facility uses this provider profile identifier to retrieve the created provider profile from the EMRS. The facility verifies the correctness of the retrieved provider profile, and causes it to be stored in the IMS, where it and its contents becomes available for retrieval along with other identity information stored for the worker by the IMS.

By performing in some or all of the ways discussed above, the facility shifts significantly earlier the time at which new providers are active in an EMRS, and thus the time when these providers can fulfill their work responsibilities. The facility also reduces the level of manual effort needed to create EMR provider profiles, and reduces the erroneous information contained by these provider profiles.

Also, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with less latency, and/or preserving more of the conserved resources for use in performing other tasks or additional instances of the same task. As one example, by automating the process of creating provider profiles, the facility reduces the level of processing resources and network bandwidth required to create provider profiles in a way that is more manual.

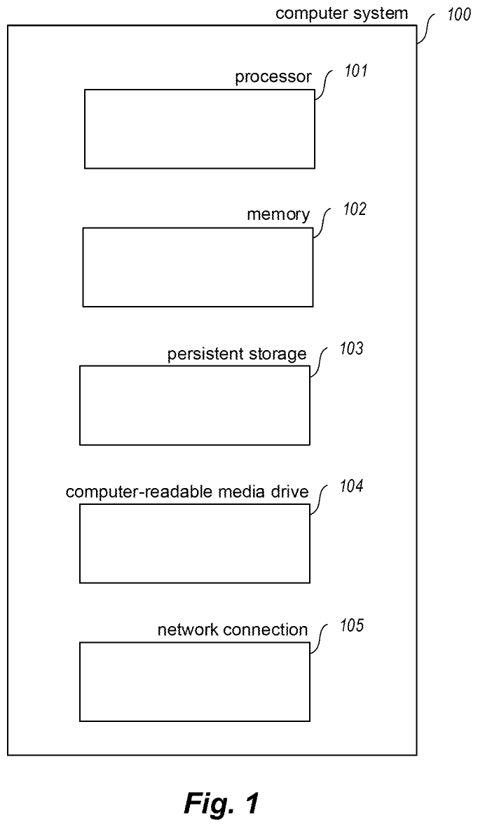

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a central processing unit (“CPU”) 101 for executing computer programs; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

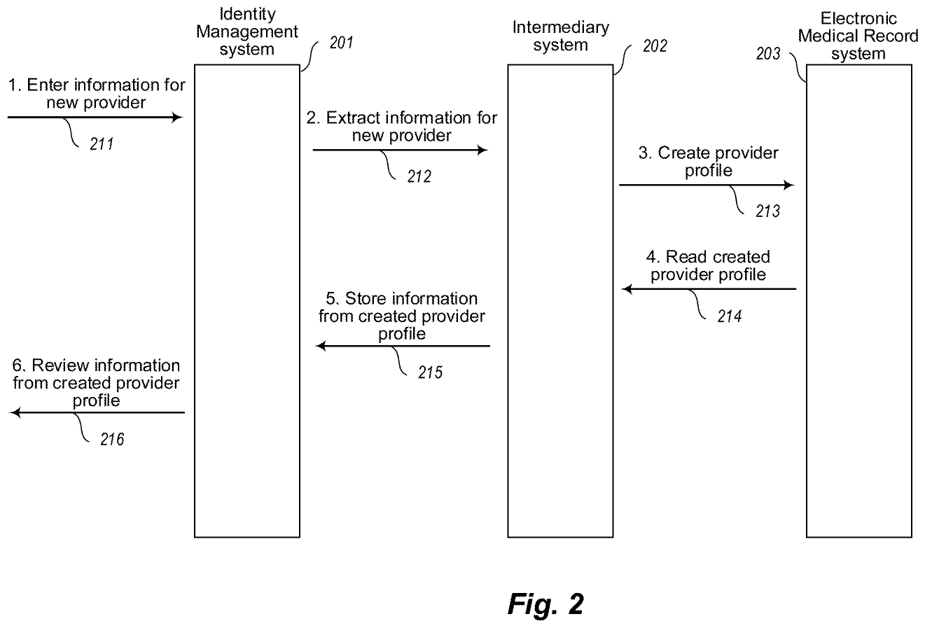

FIG. 2 is a data flow diagram showing a process performed by the facility in some embodiments in order to create provider profiles. First, information 211 for a new provider is entered into an identity management system (IMS) 201. In some embodiments, this entry is manual. In some embodiments, this entry is partly or fully automated. Next, the facility extracts information for the new provider from the identity management system to an intermediary system 202. The intermediary system 202 sends a request 213 to create a new provider profile for the new provider. In some embodiments, the request contains all of the information to be included in the created provider profile. In some embodiments (not shown), in response to the request, the electronic medical record system returns to the intermediary system 202 a provider profile identifier used to access the created provider profile within the electronic medical record system.

Next, the intermediary system 202 reads the created provider profile 214 from the electronic medical record system, such as by using the provider profile identifier returned by the electronic medical record system. The intermediary system then stores information 215 from the created provider profile in the identity management system. At any subsequent time, a user can review the information 216 from the created provider profile as stored in the identity management system.

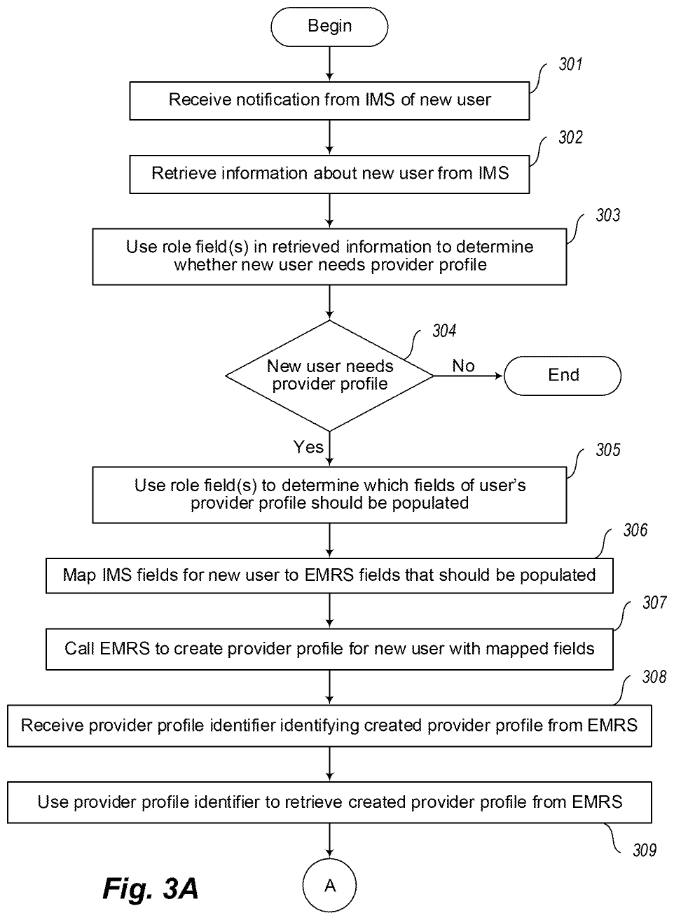

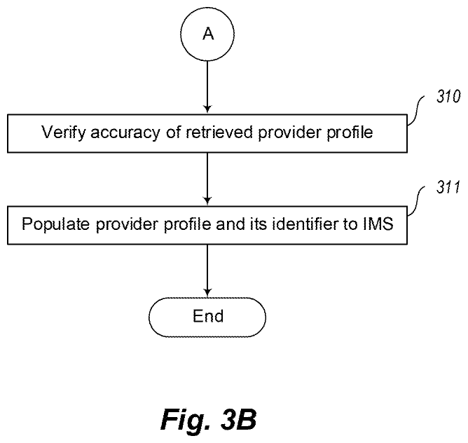

FIGS. 3A-B is a flow diagram showing a process performed by the facility in some embodiments to create a provider profile. In act 301, the facility receives a notification from the IMS that information about a new user has been added to the identity management system. In some embodiments, the facility causes the notification to be generated by an IMS server based upon information added to an IMS database. In various embodiments, the facility solicits this notification using, for example, a hook or event registration resource of the IMS server, and/or scripts or other code written specifically to implement the facility that runs on the IMS server or elsewhere. In act 302, the facility retrieves information about this new user from the IMS. In various embodiments, scripts or other code implementing act 302 execute on the IMS server, or a separate computer system. In act 303, the facility uses one or more role fields in the information retrieved in act 302 to determine whether a provider profile should be created for this new user. In act 304, if it is determined that a provider profile should be created for the new user in act 303, then the facility continues in act 305, else this process concludes. In act 305, the facility uses one or more role fields among the IMS information to determine which fields of the provider profile to be created for the user should be populated.

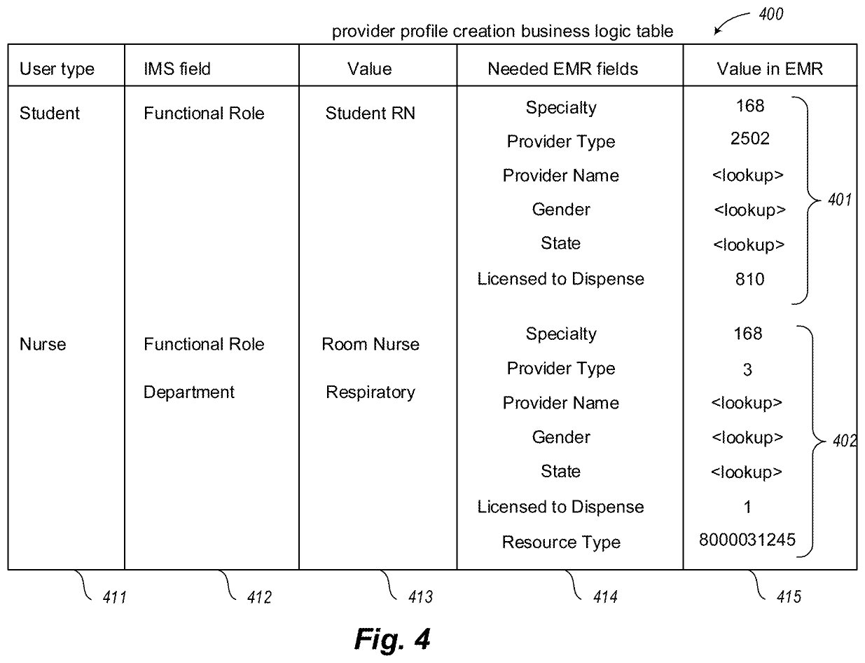

In some embodiments, the facility uses provider profile creation business logic to perform acts 303-305. FIG. 4 is a table diagram showing sample contents of a provider profile creation business logic table used by the facility in some embodiments. The provider profile creation business logic table 400 is made up of entries, such as shown entries 401, 402, and 403. As shown, each entry is divided into the following columns: a User type column 411 showing a category of user identified to the identity management system to which the entry applies; an IMS field column 412 showing a field of the identity management system whose values are to be evaluated in deciding whether a provider profile should be created and which EMR fields of the provider profile should be populated; a Value column 413 showing a value of the IMS field contained by the entry to which the entry corresponds, and for which a provider should be generated and particular EMR should be populated; and a Needed EMR fields column identifying any EMR fields that should be populated in the provider profile for users having the value contained by the entry for the IMS field contained by the entry. For example, entry 401 indicates that a user having user type “Student” that has the value “Student RN” in the “Functional Role” IMS field should be the subject of a new provider profile having the fields “Specialty”, “Provider Type”, “Name”, “Gender”, “State”, and “Licensed to Dispense”. As another example, entry 402 indicates that a user having the User type Nurse that has the value “Room Nurse” in the Functional Role IMS field and “Respiratory” in the Department IMS field, should be the subject of a new provider profile having the fields “Specialty”, “Provider Type”, “Name”, “Gender”, “State”, “Licensed to Dispense”, and “Resource Type”. In some embodiments, the decision to create a provider profile and the determination of which EMR fields of the provider profile should be populated is based on the User type and the values of zero or more IMS fields. In some embodiments, if an EMR field of the provider profile cannot be populated, the facility populates the EMR field using data found in one or more fields in the IMS system and/or one or more fields in one or more other systems.

While FIG. 4 and each of the table diagrams discussed below show a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed and/or encrypted; may contain a much larger number of rows than shown, etc.

Returning to FIGS. 3A-B, in act 306, the facility maps IMS fields for the new user to the EMRS fields the facility determined in act 306 should be populated. In some embodiments, the facility uses a field mapping table for this purposes.

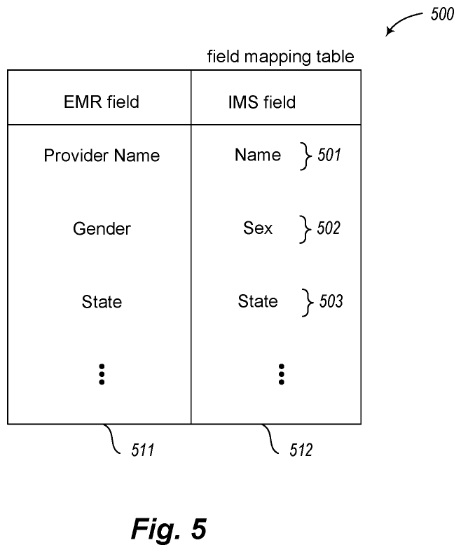

FIG. 5 is a table diagram showing sample contents of a field mapping table used by the facility in some embodiments to map between fields of the IMS and the EMRS. The field mapping table 500 is made up of rows, including shown rows 501, 502, and 503, each divided into an EMR field 511 identifying a field of the EMR, and an IMS field 512 identifying a field of the IMS that corresponds to the field of the EMR identified by the entry. For example, row 501 indicates that the EMR field “Provider Name” corresponds to the IMS field “Name”. Thus, when facility determines that the EMR field “Provider Name” should be populated for the new provider profile—as it does for new users of the User type “Student”—the facility obtains the value for so populating this EMR field from the IMS field “Name”.

Returning to FIGS. 3A-B, in act 307, the facility calls the EMRS to create a new provider profile for the new user using EMRS fields containing the data mapped from the IMS fields in act 306. In some embodiments, the call made by act 307 is against an interconnect server of the EMR to write the new provider profile to an EMR database. In some embodiments, the facility performs act 307 by calling the REST endpoint with the URI:

/2018/Core/DataUtility/ImportData/ImportData?ImportSpecificationID passing the mapped EMRS field/value pairs. In some embodiments, the facility includes a user identifier already associated with the user in the identity management system among the field/value pairs passed by the facility in the provider profile creation call in order to connect the new provider profile to the user in the identity management system. In some embodiments, the facility instructs that this IMS user identifier be stored in a Text Grouper Two field 2901 of the EMRS.

In act 308, in response to the call of act 307, the facility receives from the EMRS a provider profile identifier that identities the provider profile created by the EMRS. In act 309, the facility uses the provider profile identifier received in act 308 to retrieve from the EMRS—from the EMR database, through the EMR interconnect server—the created provider profile. In some embodiments, the facility performs act 309 using a RunQuery API of the EMRS, a SOAP endpoint having the URI:
/wcf/Epic.Core.Reporting.Services/Sql.svc
   In some embodiments, the run query passes in the same Text Grouper 2 identifier utilized to create the provider profile and returns the following attributes:
PROV_ID_1
CID_11
RPT_GRP_TWO_2901
CLIN_TITL_C_5
IS_VERIFIED_C_6
PROV_TYP_C_30
PROVIDER_TYPE_C_1040
PROV_NAME_2

In act 310, the facility verifies the accuracy of the provider profile retrieved in act 309. In act 311, the facility populates the provider profile and its identifier to the EMS for the new user, such as by using the identity management system's user identifier for the new user. In some embodiments, the facility performs act 311 by calling an API on an IMS server in order to write the data to an IMS database. After act 311, the steps conclude.

Those skilled in the art will appreciate that the acts shown in FIG. 5 may be altered in a variety of ways. For example, the order of the acts may be rearranged; some acts may be performed in parallel; shown acts may be omitted, or other acts may be included; a shown act may be divided into sub-acts, or multiple shown acts may be combined into a single act, etc.

In some embodiments, the facility performs a bulk read of provider profiles from the EMRS, and reformats this data into a common format for loading into the IMS. In some embodiments, the facility does so by using a script executing on a script server to read the data from the EMRS data store, transform the data as appropriate, and write it to a custom table within the IMS DB with the following columns:
PROV_ID_1
CID_11
PROV_ID_1
PROV_NAME_2
CLIN_TITL_C_5
IS_VERIFIED_C_6
CNCT_NUM_10
PROV_ABB_25
PROV_TYP_C_30
STAT_C_35
CONTACT_COMMENT_38
DEPT_C_40
INACT_CAD_DEPT_41
REFERRAL_TYPE_C_45
INTERNAL_REF_C_190
SERVICE_DEFAULT_C_800
ALWD_SVC_C_801
ATTND_PROV_ALL_SA_C_851
ATEND_PROV_REVK_C_854
ATTEND_PROV_CER_ID_857
PROVIDER_TYPE_C_1040
PROV_SPECIALTY_C_1050
DOCTORS_DEGREE_1060
IS_ENC_PROV_C_1081
IS_SUP_PROV_C_1082
IS_SUP_PROV_REQ_C_1083
DFLT_TRTMNT_REL_2600
RPT_GRP_ONE_2900
RPT_GRP_TWO_2901
RPT_GRP_SIX_C_2905
REVENUE_DEPT_ID_2952
SURGICAL_REC_TYPE_C_5000
RESOURCE_TYPE_ID_5003
LICENSE_DISPLAY_C_6000
IS_EC_PROV_C_8020
RSLT_ROUT_TYPE_C_8114
RESULTS_DEPT_C_8115
IS_MEDS_AUTH_PROV_C_8210
IS_PHARMACIST_C_8215
IS_ORDS_AUTH_PROV_C_8220
PREFRD_COMM_MTHD_C_8350
ADDRESS_LINE_1_21010
SECONDARY_PHONE_21100
SECONDARY_EMAIL_21110
HOME_HEALTH_DIS_C_27000
HH_VO_IB_USER_C_27010
HH_CAN_SIGN_VO_C_27020
NOTE_SVC_DEF_C_34700
ALLOWED_CLIN_SVC_C_34701
IP_DEFAULT_TT_REL_C_34825
INPT_LICENSURE_C_34850
INP_DISCIPLINE_ID_34900
IS_JP_ORD_PROV_C_34920
IS_OP_ORD_PROV_C_34921
ED_PROV_C_49000
CAN_BE_SUPER_C_49100
NEEDS_SUPERVISOR_C_49200
LAB_FAX_NUMBER_51000
   With the data stored in the IMS DB, the read portion of can use standard JDBC commands to read in all of the provider profiles within the EMRS environment.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, the method comprising:
retrieving from an identity management system information about a worker;
determining, based upon the information retrieved from the identity management system, that a provider profile should be created for the worker in an electronic medical record system;
determining, based upon the information retrieved from the identity management system, a set of provider profile fields that should be populated for the worker in the electronic medical record system;
causing a provider profile to be created for the worker in the electronic medical record system; and
for each of the determined set of provider profile fields:
mapping between the provider profile field and a field among information maintained for the worker by the identity management system; and
causing the determined field of the created provider profile to be populated with the mapped field among information maintained for the worker by the identity management system.

2. The method of claim 1 wherein the provider profile is a schedulable epic resource.

3. The method of claim 1, further comprising:
retrieving the created provider profile from the electronic medical record system; and
comparing the retrieved provider profile with the information maintained for the worker by the identity management system to validate the retrieved provider profile.

4. The method of claim 3 wherein the retrieving comprises calling a RunQuery API.

5. The method of claim 1 wherein the causing comprises calling an ImportData?ImportSpecificationID API.

6. The method of claim 1, further comprising accessing business rules that identify provider profile fields that should be populated for workers based upon information maintained for them by the identity management system.

7. The method of claim 1, wherein the causing the provider profile to be created is performed via a first channel with the electronic medical record system,
and wherein causing the provider profile fields to be populated is performed via the first channel with electronic medical record system,
the method further comprising causing to be exporting a batch of multiple provider profiles from the electronic medical record system using a second channel distinct from the first channel.

8. The method of claim 7 wherein the second channel is a JDBC channel.

9. One or more memories collectively storing a field population data structure relating to resources, the data structure comprising:
a plurality of entries, each entry comprising:
information identifying one or more classes of resources; and
information identifying one or more data items to be populated in each resource profile created for resources among the one or more identified classes,
such that the contents of the data structure are usable to determine which data items are to be populated when creating a resource profile for a particular resource, based upon the class of the resource.

10. The one or more memories of claim 9 wherein the resources to which the field preparation data structure relates are workers.

11. The one or more memories of claim 9 wherein, for least a portion of the plurality of entries, the information identifying one or more data items to be populated in each resource profile identifies data items to be populated in an electronic medical record provider profile.

12. The one or more memories of claim 9 wherein, for each of at least a portion of the plurality of entries, the information identifying one or more classes of resources identifies those one or more classes of resources with respect to information about resources contained by an identity management system.

13. The one or more memories of claim 12 wherein, for least a portion of the plurality of entries, the information identifying one or more data items to be populated in each resource profile identifies data items to be populated in an electronic medical record system provider profile,
and wherein the data structure further comprises:
a second plurality of entries, each of the second plurality of entries comprising:
information identifying a data item in the electronic medical record provider profile; and
information identifying a corresponding data item in the identity management system,
such that the contents of the data structure are usable to transform information about a resource in the identity management system into information about a corresponding provider profile in the electronic medical record system.

14. One or more memories collectively having contents configured to cause a computing system to perform a method, the method comprising:
retrieving from an identity management system information about a worker;
determining, based upon the information retrieved from the identity management system, that a user profile should be created for the worker in a software system;
determining, based upon the information retrieved from the identity management system, a set of user profile fields that should be populated for the worker in the software system;
causing a user profile to be created for the worker in the software system; and
for each of the determined set of user profile fields:
mapping between the user profile field and a field among information maintained for the worker by the identity management system; and
causing the determined field of the created user profile to be populated with the mapped field among information maintained for the worker by the identity management system.

15. The one or more memories of claim 14, the method further comprising:
retrieving the created user profile from the software system; and
comparing the retrieved user profile with the information maintained for the worker by the identity management system to validate the retrieved user profile.

16. The one or more memories of claim 14, the method further comprising accessing business rules that identify user profile fields that should be populated for workers based upon information maintained for them by the identity management system.

17. The one or more memories of claim 14, wherein the causing the user profile to be created is performed via a first channel with the software system,
and wherein causing the user profile fields to be populated is performed via the first channel with software system,
the method further comprising causing to be exporting a batch of multiple user profiles from the software system using a second channel distinct from the first channel.

18. The one or more memories of claim 17 wherein the second channel is a JDBC channel.
